(19) United States
(12) Patent Application Publication
(10) Pub. No.: US 2017/0042746 A1
(43) Pub. Date: Feb. 16, 2017

(54) UNDERGARMENT AND RELATED METHODS OF SECURING AND DISPOSAL OF SAID UNDERGARMENT

(71) Applicants: Fredrica V. Coates, Winston-Salem, NC (US); Rachel H. Star, Chicago, IL (US)

(72) Inventors: Fredrica V. Coates, Winston-Salem, NC (US); Rachel H. Star, Chicago, IL (US)

(21) Appl. No.: 15/267,593

(22) Filed: Sep. 16, 2016

Related U.S. Application Data
(63) Continuation-in-part of application No. 14/825,945, filed on Aug. 13, 2015.

(51) Int. Cl. A61F 13/49; A61F 13/56; A61F 13/496

(52) U.S. Cl. CPC A61F 13/49011; A61F 13/496; A61F 13/565

(57) ABSTRACT

An undergarment includes at least one panel of stretchable loop material. The panel of stretchable loop material is connected to a main body of the undergarment such that it can stretch around one side of wearer's waist or hip. The stretchable loop material is configured such that it can releasably connect to a plurality of engaging elements. The engaging elements can be hooks of a hook and loop fastening system. The undergarment is configured such that it can be easily concealed beneath clothing and can be easily positioned and removed by a wearer. A method of disposing an undergarment includes rolling or folding the main body of the undergarment and tying panels of stretchable loop material around the rolled or folded portion to encase the rolled or folded portion between the panels of stretchable loop material.

### CROSS REFERENCE TO RELATED APPLICATION

This application is a Continuation-In-Part application and claims priority to Nonprovisional application, U.S. Ser. No. 14/825,945 filed Aug. 13, 2015.

### BACKGROUND OF THE INVENTION

Undergarments, especially disposable or semi-disposable undergarments, are often used by individuals in order to capture urinary leakage attributable to incontinence, menstrual flow, as well as to capture semi-solid and solid waste discharged from the large intestine. Incontinence can be attributed to a variety of medical conditions. Individuals with such conditions may use disposable undergarments to capture involuntary urinary leakage. Such individuals require disposable undergarments that can be worn under clothing.

Known disposable undergarments suffer from many disadvantages. Known disposable undergarments are often bulky, poorly fitting, and inconvenient for use in an active lifestyle and create problems during disposal. Many known disposable undergarments are of the pull-on type. As such, the disposable undergarment may require a wearer to remove his or her pants in order to remove and replace the disposable undergarment. This can be a time consuming and cumbersome activity that does not work well for individuals with active or busy lifestyles. In addition, known disposable undergarments are often cumbersome due to the fact that they are designed to retain both solid and liquid wastes and are also designed to be one-size fits-all products. Known undergarments do not conform to a wearer's body and can be seen through a wearer's clothing due to the attachment schemes, bulky absorbent cores and general over-engineering of the products. There exists a need, therefore, for a disposable undergarment that can be easily concealed, easily removed and replaced during normal daily life and that can accommodate a variety of body types and sizes.

Known disposable undergarments also can be difficult to dispose of easily and sanitarily after they have become soiled. It can be difficult to retain the captured wastes in the soiled undergarment once it has been removed from a wearer. There exists a need for a disposable undergarment that can retain bodily wastes and can be packaged or configured to retain such wastes from a point of removal to a point of disposal.

### SUMMARY OF THE INVENTION

The instant disclosure describes various embodiments of an undergarment. In a first embodiment, an undergarment includes at least one panel of stretchable loop material. The panel of stretchable loop material is connected to a main body of the undergarment such that it can stretch around one side of a wearer's waist or hip. The stretchable loop material is configured such that it can releasably connect to a plurality of engaging elements. The engaging elements can be hooks of a hook and loop fastening system. The stretchable loop material can be a knit fabric that includes spandex yarn.

In a second embodiment, the undergarment includes a first panel and a second panel of stretchable loop material connected to opposite sides of the main body of the undergarment.

In a third embodiment, the height of the first flap is at least twice as long as its width.

In another embodiment, the undergarment includes a main body with an absorbent core with a first and second longitudinal side. A first tab is connected to the main body on the first longitudinal side and includes a first attachment area with a plurality of engaging elements. The undergarment also includes a first flap extending from the main body on the first longitudinal side. The first flap is made of stretchable loop material that is configured to releasably and directly connect to the first tab by engaging with the plurality of engaging elements.

In another embodiment, any location on the first flap's body-facing surface can engage with the plurality of engaging elements.

In another embodiment, the first tab and the first flap are configured to permit the undergarment to be worn by wearers with variations in weight of over 20 pounds.

In another embodiment, the undergarment further includes a second tab and a second flap both connected to the main body on the second longitudinal side. The second flap is made of stretchable loop material configured to releasably and directly connect to the second tab by engaging with a plurality of engaging elements on the second tab.

### BRIEF DESCRIPTION OF THE DRAWINGS

Certain embodiments are shown in the drawings. However, it is understood that the present disclosure is not limited to the arrangements and instrumentality shown in the attached drawings, wherein:

FIG. 1 is an illustration of one embodiment of a disposable undergarment of the present disclosure.

FIG. 2 is an illustration of the garment-facing side of the embodiment of FIG. 1.

FIG. 3 is a side view of one embodiment of a disposable undergarment of the present disclosure on a wearer.

FIG. 4 is a front view of one embodiment of a disposable undergarment of the present disclosure on a wearer.

FIG. 5 is an illustration of another embodiment of a disposable undergarment of the present disclosure.

FIG. 6 is an illustration of the garment-facing side of the embodiment of FIG. 5.

FIG. 7 is an illustration of one embodiment of the undergarment in the process of being prepared for disposal.

FIG. 8 is an illustration of one embodiment of the undergarment in a tied position ready for disposal.

FIG. 9 is a perspective view of another embodiment of a disposable undergarment.

FIG. 10 is a cross-section view of the absorbent structure utilized in the disposable undergarment shown in FIG. 9.

FIG. 11 is a top view of the disposable undergarment shown in FIG. 9 when in a closed configuration with the first and second members of the pair of lateral sides secured together by the pair of attachment members.

FIG. 12 is a perspective view of another embodiment of a disposable undergarment having at least two elastic strands located in the stretchable loop material which are aligned parallel to the hip opening and which are positioned above the first and second ends of the absorbent structure.

FIG. 13 is a perspective view of still another embodiment of a disposable undergarment having at least two elastic strands which extend completely around the hip opening.

FIG. 14 is a front view of still another embodiment of a disposable undergarment constructed entirely of a stretchable loop material and having a removable absorbent structure.

FIG. 15 is a back view of the absorbent structure which would be removably secured to the disposable undergarment shown in FIG. 14.

FIG. 16 is a front view of the absorbent structure which would be removably secured to the disposable undergarment shown in FIG. 14.

FIG. 17 is a partial cross-sectional view of the disposable undergarment shown in FIG. 14 taken along line 17-17.

### DETAILED DESCRIPTION OF THE INVENTION

For the purposes of promoting and understanding the principles disclosed herein, references are now made to the preferred embodiments illustrated in the drawings and specific language is used to describe the same. It is nevertheless understood that no limitation of the scope of the invention is thereby intended. Such alterations and further modifications in the illustrated device and such further applications of the principles disclosed as illustrated herein are contemplated as would normally occur to one skilled in the art to which this disclosure relates.

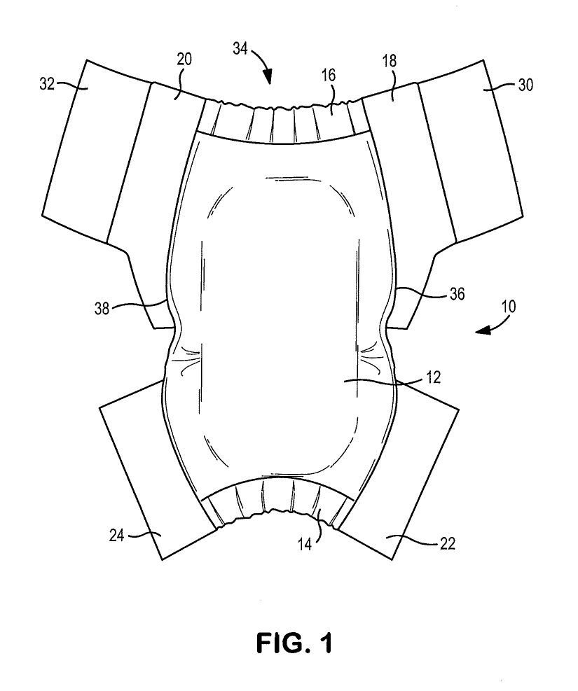

FIGS. 1-4 show one embodiment of disposable undergarment 10. Disposable undergarment 10 may include a main body 34, first and second extensions 18, 20, first and second tabs 22, 24 and first and second flaps 30, 32. Disposable undergarment is generally shaped and contoured to be worn dose to a wearer's body such that it can be concealed while still capturing leaks, bodily fluids and waste. Main body 34 is the central portion of disposable undergarment 10 and includes absorbent core 12 that serves the purpose of capturing fluid and preventing its escape from disposable undergarment 10. Various structures and layers of materials can be used to construct main body 34 and absorbent core 12.

The main body may include a top sheet and one or more back sheets. The layers of material can be paper, plastic or other suitable natural or synthetic fabrics. Such materials can be suitable nonwoven materials. The materials closest to a wearer's body are often made of breathable or permeable materials such that wastes are permitted to pass through such materials and are kept away from a wearer's skin. Other outer layers and materials can be made from impermeable materials such that bodily wastes are not permitted to escape or otherwise leak from the undergarment. Elastic bands or other materials can be fixed between the layers of material or to the layers of material to provide elasticity, gathers or other features to conform the disposable garment 10 to the body of a wearer. The absorbent core 12 may include one or more acquisition and liquid absorption layers that are intended to capture and hold bodily fluids. Known methods of connecting the various layers of main body 34 may be used to construct disposable undergarment 10 such as thermal bonding, adhesive, ultrasonic sealing and various stitching methods.

Main body 34 may also include front waist portion 14 and rear waist portion 16. As can be appreciated, in use, front waist portion 14 is configured to fit against the front of a wearer's waist area. Rear waist portion 16 is configured to fit against the rear waist of a wearer under the wearer's lower back. As stated earlier, disposable undergarment 10 is low-rise such that it can be concealed under a wearer's clothing. As such front waist portion 14 and rear waist portion 16 are configured to be positioned on a wearer's body under and away from the waist region of wearer's clothing. Front waist portion 14 and rear waist portion 16 may include elastic bands, elastic strips or other features to contour disposable undergarment 10 against the body of a wearer. Furthermore, the elasticity provided at front waist portion 14 and rear waist portion 16 can provide the benefit of permitting disposable undergarment 10 to be worn by wearers of various shapes and sizes while still reliably capturing and retaining bodily fluids. Elastic bands, strips or other extensible members can be embedded between layers of main body 34 at front waist portion 14 and rear waist portion 16 or can be connected at these locations via various methods of attachment, as discussed above, such as thermal bonding, adhesive, ultrasonic sealing or stitching.

Along the longitudinal sides of main body 34 (i.e., in a direction extending between front waist portion 14 and rear waist portion 16) various members or panels may be attached to assist with the attachment of disposable undergarment 10 to the body of a wearer. In the example shown in FIGS. 1-4, disposable undergarment 10 includes first and second extensions 18, 20, first and second flaps, 30, 32 and first and second tabs 22, 24. First and second extensions 18, 20 are connected to main body 34 near rear waist portion 16 and are further connected to main body 34 along the longitudinal sides of main body 34 from waist portion 16 toward the center of main body 34. First and second extensions 18, 20 can be separate pieces attached to main body 34 or can be extensions of one or more layers of the material that is used to construct the main body of disposable undergarment 10. The first and second extensions 18, 20 are preferably made of an elastic material such as an extensible laminate or a breathable partial laminated nonwoven material. Other suitable materials may also be used.

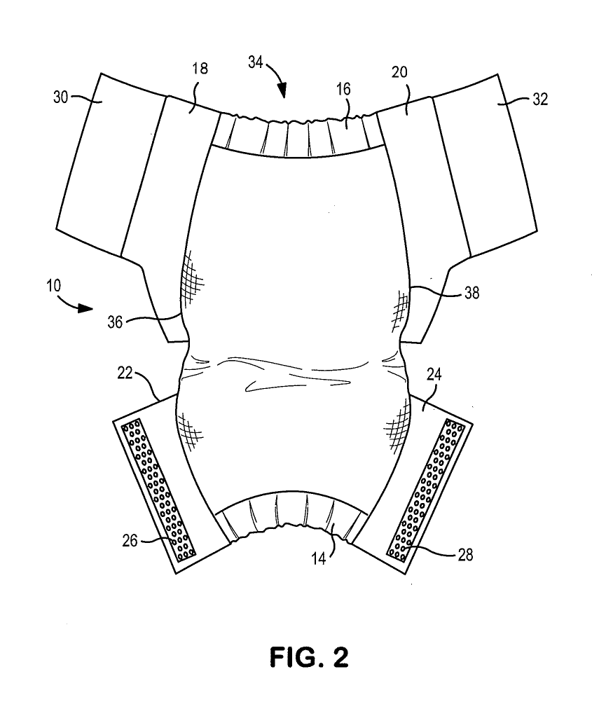

As can be seen in FIGS. 1 and 2, in this example, first and second extensions 18, 20 are connected to main body 34 at a proximal end of first and second extensions 18, 20. At distal ends of first and second extensions 18, 20, first and second flaps 30, 32 are connected. First and second flaps 30, 32 are second pieces of material connected to and extending in a transverse direction away from main body 34. First and second flaps 30, 32 have a width that is measured in a direction that is generally parallel to a top edge of rear waist portion 16 and a height that is measured in a direction that is generally perpendicular to the width. In this example, the height of first flap 30 is at least approximately twice as long as the width. While various sizes of disposable undergarment 10 can be constructed, in one example configuration, first and second flaps 30, 32 are approximately 55-65 mm wide and 100-130 mm long. Other sizes and shapes of first and second flaps 30, 32 can also be used.

First and second flaps 30, 32 can be connected to first and second extensions 18, 20 via any suitable means. Suitable methods of attachment may include, but are not limited to, adhesive, stitching, thermal bonding, ultrasonic sealing and the like. In one preferred embodiment, first and second flaps 30, 32 are secured via an over lock stitch to first and second extensions 18, 20. First and second flaps 30, 32 are preferably made of a stretchable loop material. Stretchable loop material is elastic and provides the advantage of contouring the flaps to a wearer's body and accommodating a wide variety of wearer body types and shapes. In one example, first and second flaps 30, 32 are made of a stretchable loop material that is a woven or knit material that is capable of being extended to at least twice its initial length. In other examples, the stretchable loop material can be extended to 220% of its original, non-extended length. Stretchable loop material additionally has the property that it can securely engage to the hook portion of a typical hook-and-loop fastening system. In this manner, and as will be described, first and second flaps 30, 32 can directly engage to an engaging element of a hook and loop fastening system. This property allows first and second flaps 30, 32 to be made of a single material without the need for manufacturing the flaps from multi-layers of material or to connect a separate fastening mechanism or a separate piece of loop material to the flap. This property of the stretchable loop material of first and second flaps 30, 32 also permits the engaging of the flaps at any position along its entire surface. The stretchable loop material permits such engagement, disengagement and re-engagement a significant number of times. These properties provide a significant benefit in providing flexibility, adjustment and comfort for a wearer.

Stretchable loop material with the properties described above can be constructed of different materials using different types of fabrics and threads. In one example stretchable loop material, the material is formed from a stitch bonded fabric that includes a non-bonded fibrous layer in which elastic yarns, such as spandex elastomeric yarns, are stitched to create rows in the fabric with differing abilities to stretch. In this example, the stretchable loop material provides for stretch of 190% or more in one row and for stretch of 60% or more in a second row. Substantially perpendicularly from the direction of stretch described above, this example stretchable loop material also provides for 80% to 90% stretch in a second direction as well. This example stretchable loop material has a significant overall stretch in the first direction as well as no more than 90% stretch in a substantially perpendicular direction.

In a preferred embodiment of the above example stretchable loop material, two types of threads are knitted into the substrate fabric. A first spandex thread that is covered with a texturized nylon can be used in addition to a second polyester thread. The first spandex thread, with a texturized nylon cover, is knitted into the substrate fabric at predetermined intervals in one direction using a simple chain stitch. The second polyester thread can then be knitted into the substrate fabric in one direction using a tricot stitch. These two types of threads can give the stretchable loop fabric a striped or banded appearance.

As discussed above, one of the threads or yarns used in the stretchable loop material can be textured. Textured yarn or thread is subjected to a procedure to increase the volume and the elasticity of the thread or yarn. Textured yarn exhibits twists, crimps, or other distortions along its filaments. In addition to increased elasticity, textured yarns and threads can impart other advantageous properties to fabrics in which it is woven, stitched or otherwise used. One such advantage can be the ability to engage with the hook portion of a hook and loop fastening system. The twists, crimps or other distortions give the fabric into which the textured yarn or thread is incorporated a “fuzziness” that can increases its ability to engage and re-engage the hook of a hook-and-loop fastening system.

In one example of disposable undergarment 10, first and second flaps 30, 32 are made of a stretchable loop material such as a knitted fabric of the same construction as a bed skirt knitted fabric available from Xymid, LLC of Midlothian, Va. Another example of a stretchable loop material is a polyester knitted fabric sold as a cover for a mattress by Sinomax USA, Inc. of Houston, Tex. These materials are examples of materials that provide flexibility and extendibility and are able to securely engage to the hook portion of a hook and loop fastening system.

In another preferred example of stretchable loop material, the fabric is a knitted fabric using spandex elastic yarn combined with polypropylene. The example stretchable loop fabric may also have one or more layers that can be knitted from the same type of yarn or from different types of yarns, such as from yarns of polyester. Another example knitted fabric stretchable loop material is made of polyester, nylon and spandex yarns. Other suitable woven and knit materials can also be used. Such suitable stretchable loop material's capability to engage the hook portion of a hook and loop fastening system can be further improved by subjecting the material to heat treatment prior to manufacturing of the disposable undergarment. Such heat treatment can include repeated washing and drying of the material on high heat prior to manufacturing of the disposable undergarment. Heat treatment of the material can increase the force required to disengage the material from a hook portion of a hook and loop fastening system.

As further shown in FIGS. 1-4, disposable undergarment 10 can also include first and second tabs 22, 24. The first and second tabs 22, 24 are connected to main body 34 at a location at or near front waist portion 14. First and second tabs 22, 24 may be elongated pieces of material as shown and may extend longitudinally along the longitudinal sides of main body 34 from front waist portion 14 toward the center of main body 34. In one example, the length of first tab 22 can be generally the same size as the height of first flap 30. First and second tabs 22, 24 can be separate pieces of material joined to main body 34 or can be extended portions of the one or more layers of material that are used to construct main body 34. In one example, first and second tabs 22, 24 are pieces of flexible material such as an extensible laminate. Other suitable materials may also be used.

As further shown in FIGS. 1 and 2, first and second tabs 22, 24 may include first and second attachment areas. First and second attachment areas are portions of first and second tabs 22, 24 to which first and second flaps 30, 32 can be connected. In one example, first and second attachment areas of the first and second tabs 22, 24 can be hook portions of a typical hook and loop fastening system. A piece of a hook portion of a hook and loop fastening system can include a base with a plurality of engaging elements extending therefrom. This piece of a hook portion can be connected to first and second tabs 22, 24 via any suitable attachment method such as, but not limited to, adhesive, thermal bonding, ultrasonic sealing or stitching. In one example, first and second attachment areas are connected to and extend along a distal edge of first and second tabs 22, 24.

As shown in FIG. 2, the engaging elements of first and second attachment areas of the first and second tabs 22, 24 are located on the garment-facing surface of first and second tabs 22, 24 that is on the surface that faces the clothes of a wearer and away from the body of a wearer. In this manner, first and second attachment areas of the first and second tabs 22, 24 are exposed when disposable undergarment 10 is positioned on a wearer and are readily available for a wearer to secure first and second flaps 30, 32.

In one example, first and second attachment areas are pieces of hook material from a hook and loop fastening system that are approximately 10-15 mm wide by 90-100 mm long. In other examples of disposable undergarment 10, first and second tabs 22, 24 may include one or more first and second attachment areas. Instead of or in addition to first and second attachment areas of the first and second tabs 22, 24, as shown on FIG. 2, first and second tabs 22, 24 may include a plurality of attachment areas. Several individual pieces of the hook portion of a hook and loop fastening system can be connected to first and second tabs 22, 24. The addition of more than one attachment area can provide the advantage of increased versatility and permit disposable undergarment 10 to be fitted to a variety of body shapes and sizes. In still another example, additional attachment areas can be placed on main body 34 at the garment-facing side of main body 34 to provide additional areas where first and second flaps 30, 32 can directly attach. The attachment areas can be in a variety of shapes and patterns so as to provide further variations on the fit and positioning of disposable undergarment 10.

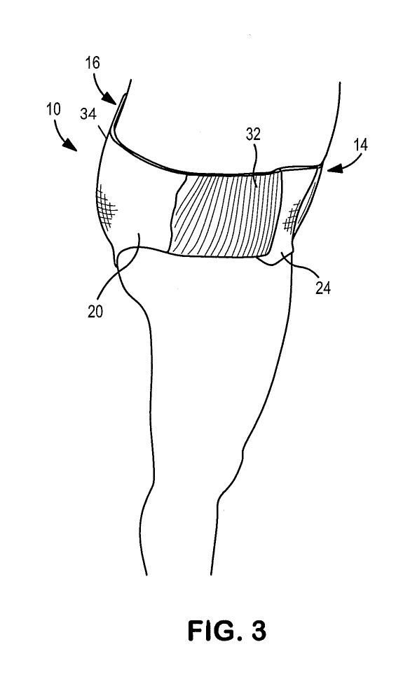

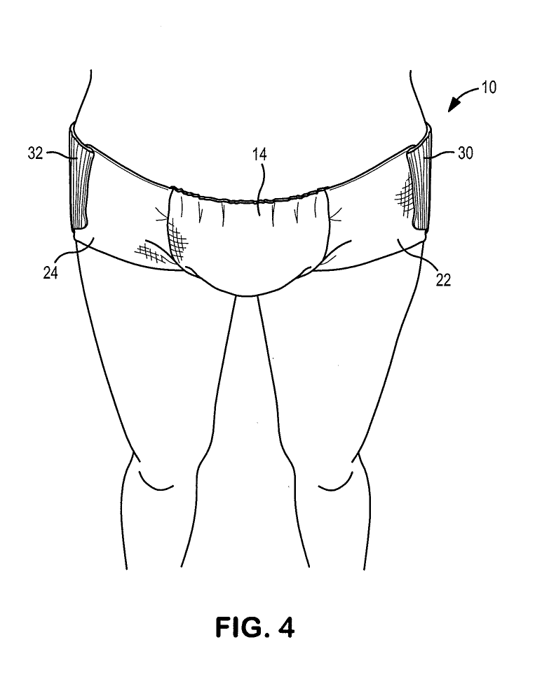

FIGS. 3 and 4 show one embodiment of disposable undergarment 10 secured to a wearer. As can be seen, disposable undergarment 10 is a low-profile or low-rise product, such that it can easily be concealed beneath clothing. Additionally, it is removable, disposable and capable of fitting a wide variety of wearers including wearers, whose respective weights may differ by 20 pounds or more. When secured on a wearer, disposable undergarment 10 wraps around the waist and hip area of a wearer. In doing so, the upper edges of first and second extensions 18, 20, first and second flaps 30, 32 and first and second tabs 22, 24 coupled with the upper edges of front waist portion 14 and rear waist portion 16 circumferentially encompass the waist area of the wearer, see FIG. 3. The main body portion 34, the upper edges of front waist portion 14 and rear waist portion 16 occupy a relatively small portion of the circumferential waist of disposable undergarment 10. The majority (or more than half) of a wearer's waist is not touched by the main body 34. In other embodiments and sizes of undergarment 10, the main body portion 34 can occupy even less of the circumferential waist of disposable undergarment 10. In one such example, the main body occupies only one-third of a wearer's waist. The majority of a wearer's waist is wrapped by the first and second extensions 18, 20, first and second flaps 30, 32 and first and second tabs 22, 24. The extensible nature of these elements provides a form-fitting and unobtrusive undergarment. This structure of disposable undergarment 10 also provides the flexibility to secure to wearers of a variety of sizes and shapes.

Referring now to FIGS. 2, 3, and 4, the material of first and second flaps 30, 32 is configured of stretchable loop material such that it can directly secure to first and second attachment areas of the first and second tabs 22, 24. No separate piece of loop material of a hook and loop fastening system is required. The material of first and second flaps 30, 32 can directly secure to first and second attachment areas of the first and second tabs 22, 24 at any location along their surfaces. This also adds to the flexibility, fit and reliability of disposable undergarment 10. A wearer need only hold main body 34 and/or first tab 22 in position and pull or stretch first flap 30 into position and apply pressure at first attachment area 26, see FIG. 2 to secure one side of disposable undergarment 10 in position. This can be repeated on the second side of disposable undergarment 10 to fix the garment in position. For smaller or thinner wearers, first or second flap 30, 32 can be pulled into an overlapping position for a tighter fit. In such a situation, the distal end of first or second flap 30, 32 would extend over or past first and second attachment area of the first and second tabs 22, 24 such that first or second flap 30, 32 is engaged at a location on first or second flap 30, 32 nearer to its connection to first or second extension 18, 20, see FIG. 2. The fact that the stretchable loop material can engage the engaging elements at any location on its surface enables the undergarment to be worn by a wider variety of wearers including wearers with variations in weight of more than twenty pounds.

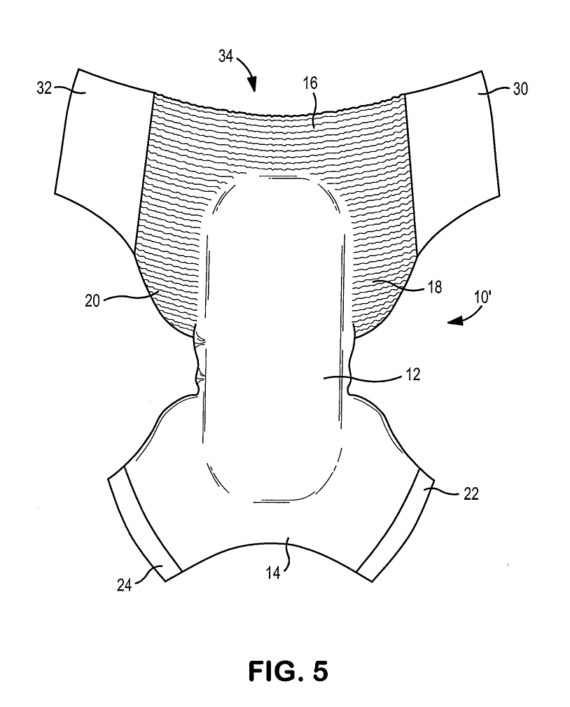

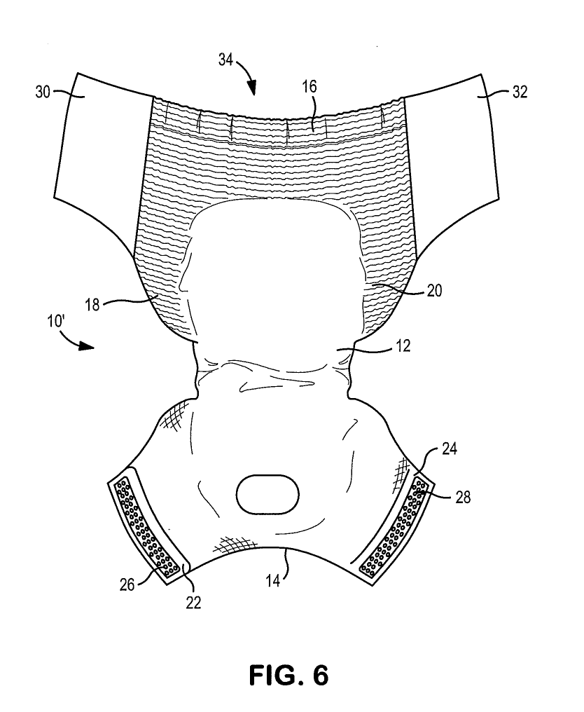

FIGS. 5 and 6 show a second embodiment of disposable undergarment 10′. As can be appreciated, many of the same elements are included in this embodiment such as first and second extensions 18, 20, first and second flaps 30, 32 and first and second tabs 22, 24. Front and rear waist portions 14, 16 are also shown as well as main body 34 and absorbent core 12. However, as can be seen, in this embodiment, front waist portion 14 is more curved in shape as are some of the edges of first and second extensions 18, 20, first and second flaps 30, 32 and first and second tabs 22, 24. Various modifications can be made to the relative sizes and shapes of the various elements described herein in an effort to conform disposable undergarment 10 to various shapes and sizes of wearers.

In still other embodiments of the disposable undergarment 10, or 10′ the various layers, flaps and attachment components may include other variations of the elements described above and can include variations in which the panels and attachments may be reversed from front to back or back to front. In another example, first and second extension 18, 20 and first and second flap 30, 32 may all be made of stretchable loop material. The stretchable loop material can also be a continuous piece of material that extends from the main body 34 across the buttocks of a wearer and extend beyond the first and second sides 36, 38 of main body 34 to create wrap portions that can be releasably attached to the first and second tabs 22, 24. In this example, since the wrap portions are made of stretchable loop material, they can be directly connected to a hook portion of a hook and loop fastening system. In other examples, first and second tabs 22, 24 can also be made of a stretchable loop material. Embodiments of disposable undergarment 10 wherein first and second tabs 22, 24 are made of stretchable loop material can enable the undergarment to fit a wider range of body sizes such as wearers with larger waistlines. As explained above, the stretchable loop material may also extend into or over portions of the main body 34 at the front waist region. Such configurations that use more of the stretchable loop material for portions of the main body 34, as well as for fastening the disposable undergarment around the waist and hips of wearer, can enable a tighter and more body-conforming undergarment.

Disposable undergarment 10 also has an improved design for easier and more sanitary disposal. Once a disposable undergarment captures bodily fluids or other wastes, it must be disposed. Some existing designs of disposable undergarments permit leakage of bodily fluids or other wastes, either during use or upon removal and disposal. Disposable undergarment 10 or the present disclosure permits a wearer to remove the used undergarment and secure the bodily fluids in the absorbent core for easy and sanitary disposal.

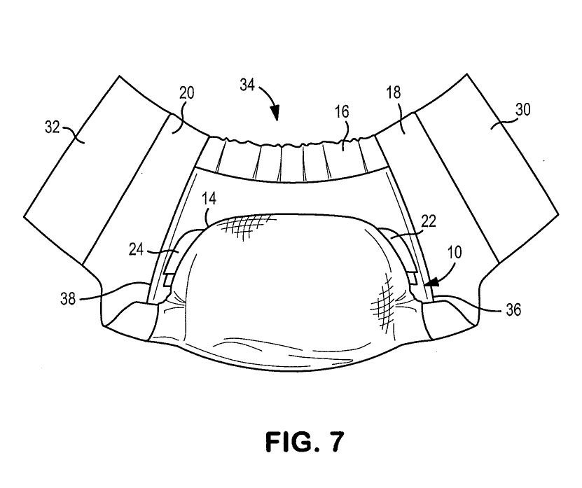

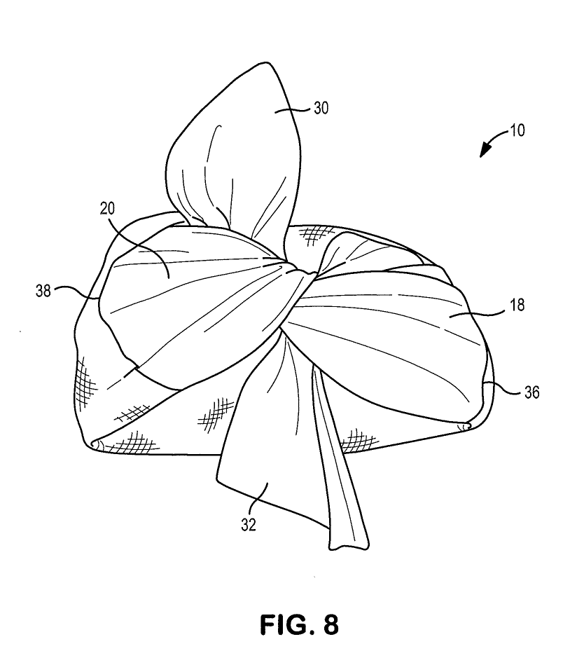

As shown in FIGS. 7 and 8, in order to dispose of the disposable undergarment 10, the front waist portion 14 can be rolled, folded or otherwise compressed toward the center of disposable undergarment 10 thus capturing the bodily fluid or waste in absorbent core 12. As shown in FIG. 7, front waist portion 14 is compressed, rolled or folded into a position substantially between first and second flaps 30, 32. First and second flap 30, 32 can then be stretched and tied with an overhand or other suitable knot toward each other. In this manner, rear waist portion 16 and first and second flaps 30, 32 wrap around and encase front waist portion and the compressed absorbent core 12 to secure the undergarment in this position for easy and sanitary disposal. The stretchable loop material of first and second flap 30, 32 has the elasticity to encompass the compressed absorbent core and to remain in a tied position as shown in FIG. 8. As can be appreciated, in other alternate embodiments of disposable undergarment 10 wherein more portions of main body, first and second extension or first and second tab are made of stretchable loop material, additional methods of disposal can also be used that wrap and tie the stretchable loop material around a compressed portion of disposable undergarment 10.

In other embodiments of the present disclosure, the undergarment is reusable and not disposable. One or more of the materials can be constructed of natural or synthetic fabrics. The reusable undergarment makes use of the stretchable loop material on first and second flaps 30, 32 that are attached to the other reusable fabrics that are used to construct the various other portions of the reusable undergarment. In this manner, the low-rise, non-bulky aspects of the undergarment as previously described can be maintained in a reusable form. The stretchable loop material on first and second flaps 30, 32 is able to directly engage the hook portion of a hook and loop fastening system on or near a front waist portion of the reusable undergarment.

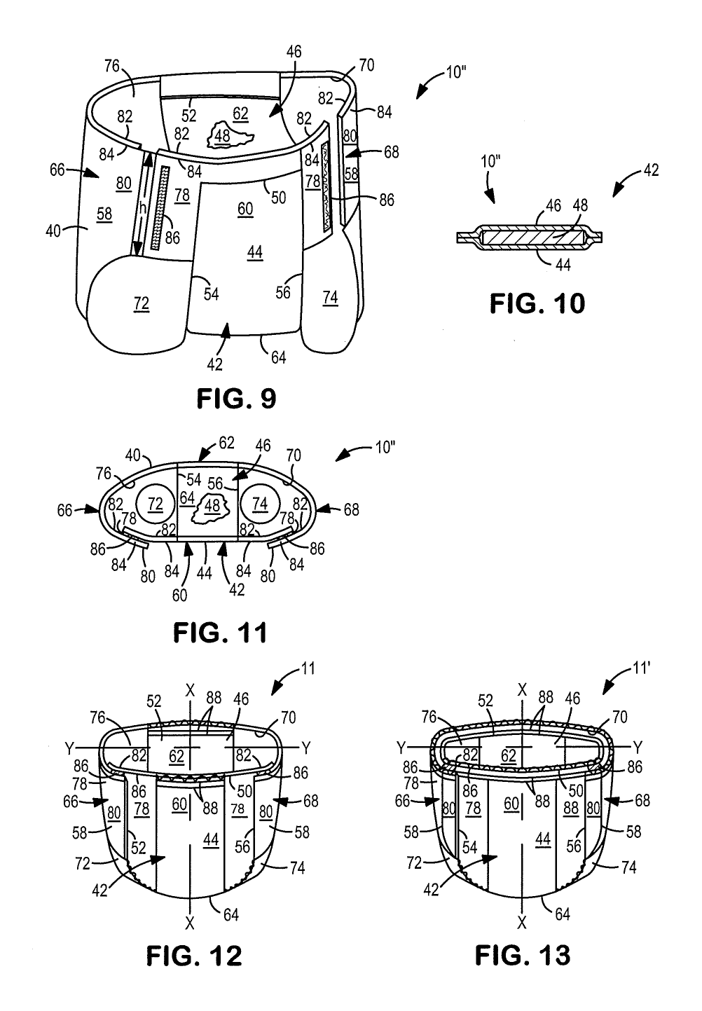

Referring now to FIGS. 9-11, another embodiment of a disposable undergarment 10″ is shown. The disposable undergarment 10″ can be worn once and then be discarded or it could be worn two or more times before it is discarded. In this embodiment, the disposable undergarment 10″ has an exterior surface 40, see FIGS. 9 and 11. The disposable undergarment 10″ is capable of being positioned on and being removed from a human torso in either a dosed configuration, see FIG. 11, or in an open configuration, see FIG. 9. By “torso” it is meant the human body excluding the head and limbs; trunk.

Referring to FIG. 10, the disposable undergarment 10″ includes an absorbent structure 42 having a liquid-impermeable outer cover 44, a liquid-permeable bodyside liner 46 secured to the outer cover 44, and an absorbent 48 positioned between the outer cover 44 and the bodyside liner 46.

Referring now to FIG. 9, the absorbent structure 42 has a first end 50, a second end 52, a first side 54 and a second side 56. The shape of the absorbent structure 42 can vary. Generally, the absorbent structure 42 has a rectangular shape but almost any other geometrical configuration could also be manufactured. For example, the absorbent structure 42 could have an hourglass shape with concave first and second sides. Another option for the absorbent structure 42 is a trapezoidal shape. By “trapezoid” it is meant a quadrilateral having two parallel sides.

Referring again to FIGS. 9 and 11, the disposable undergarment 10″ also includes a stretchable loop material 58. By “stretchable” it is meant having the ability to be stretched. The stretchable loop material 58 is capable of shrinking about 50% or more when subjected to a wash and an elevated heat cycle. This ability to shrink also enables the stretchable loop material 58 to stretch from about 150% to about 220%. The stretchable loop material 58 also has to be capable of being dyed to a color which is darker than white. This ability to be dyed to a different color is advantageous in making the disposable undergarment 10″ discrete and allowing it to appear similar to regular cloth undergarments. In addition, colored disposable undergarments can blend in with different color outer garments to conceal the fact that they are being worn. Sometimes, it is desirable that the stretchable loop material 58 is dyed to a flesh color to be inconspicuous when being worn.

The stretchable loop material 58 can include various kinds of elastic and thermoplastic materials as described supra. Typically, the stretchable loop material 58 contains elastic or is an elastic film or laminate which is capable of being stretched or elongated in at least two different directions. The stretchable loop material 58 can be a single layer or be a laminate. By “laminate” it is meant consisting of two or more layers. The stretchable loop material 58 can be a stitch-bonded nonwoven fabric. Stitch-bonded nonwoven fabrics are commercially from various sources. One such source is Xymid, LLC having an office in Midlothian, Va. Xymid, LLC has a number of U.S. patents which describe different materials having stretchable properties. Four of such U.S. patents include: U.S. Pat. Nos. 7,141,290; 7,431,975; 7,425,359 and 8,021,735 which are incorporated by reference and made a part hereof.

When the stretchable loop material 58 is a stitch-bonded nonwoven fabric it can be constructed to include a substrate which is bonded with a binder applied to the substrate in a uniform manner such that substantially no areas greater than the spaces between the stitch insertion points are devoid of binder. The binder may constitute from between about 2% to about 25% of the total weight of the sum of the binder and the substrate.

It should be noted that the stretchable loop material 58 should be constructed from a material that can be washed, dried, laundered and/or heated. For example, the material could be laundered in a common household washer and then be dried in a household dryer. The material should be capable of being washed and then dried several times without losing its stretchability. It has been found that heating the material actually increases its stretchability. Desirably, the stretchable loop material 58 can be washed and dried, i.e. laundered, at least ten times. More desirably, the stretchable loop material 58 can be washed and dried, i.e. laundered, at least fifteen times. Even more desirably, the stretchable loop material 58 can be washed and dried, i.e. laundered, at least twenty times. Most desirably, the stretchable loop material 58 can be washed and dried, i.e. laundered, over twenty times. The ability of the stretchable loop material 58 to hold up and retain its stretchability after a number of washings and drying cycles will allow the disposable undergarment 10″ to be laundered several times before it is discarded. Even though the undergarment 10″ is disposable, it does not have to be discarded after a single use or even after several uses.

The stretchable loop material 58 has a basis weight of at least 15 grams per square meter (gsm). Desirably, the stretchable loop material 58 has a basis weight ranging from between about 15 gsm to about 200 gsm. More desirably, the stretchable loop material 58 has a basis weight of from between about 15 gsm to about 175 gsm. Even more desirably, the stretchable loop material 58 has a basis weight of from between about 50 gsm to about 150 gsm. Still more desirably, the stretchable loop material 58 has a basis weight of from between about 75 gsm to about 150 gsm. Most desirably, the stretchable loop material 58 has a basis weight of greater than 100 gsm.

The stretchable loop material 58 also has a density which is at least about 0.02 grams per centimeter cubed (g/cm³). The density of the stretchable loop material 58 can range from between about 0.01 g/cm³ to about 0.15 g/cm³. Desirably, the stretchable loop material 58 has a density ranging from between about 0.02 g/cm³ to about 0.12 g/cm³. More desirably, the stretchable loop material 58 has a density ranging from between about 0.03 g/cm³ to about 0.10 g/cm³. More desirably, the stretchable loop material 58 has a density greater than about 0.05 g/cm³. Even more desirably, the stretchable loop material 58 has a density greater than about 0.07 g/cm³. Most desirably, the stretchable loop material 58 has a density greater than about 0.08 g/cm³.

As stated above, the stretchable loop material 58 is dyed, printed or treated in some fashion to change to a color darker than white. By “white” it is meant the achromatic color of maximum lightness: the color of objects that reflect nearly all light of all visible wavelengths; the complement or antagonist of black. The stretchable loop material 58 can be dyed to any color or hue known to man. By “color” it is meant that aspect of things that is caused by differing qualities of the light reflected or emitted by them, definable in terms of the observer or of the light, as: a) the appearance of objects or light sources as perceived by the individual and involving hue, lightness, and saturation for objects and hue, brightness, and saturation for light sources, b) the characteristics of light by which the individual is made aware of objects or light sources, described in terms of dominant wavelength, luminance, and purity. By “hue” it is meant the property of colors by which they can be perceived as ranging from red through yellow, green, and blue, as determined by the dominant wavelength of the light. The number of different colors is indefinite. Examples of colors useful in manufacturing the disposable undergarment 10″ include, but are not limited to: pink, purple, magenta, blue, tan, brown, black, etc. Colors that are similar to the various shades of human skin are also desirable, such as flesh colors. Softer colors are generally preferred over brighter colors.

The stretchable loop material 58 can be dyed using any known dyeing operation. By “dyeing” it is meant to impart a color to something. A number of different manual and/or commercial dyeing operations exist today. In some dyeing operations, the fabric or material can be placed in a vessel containing a bath or solution. A quantity of a particular dye is then added. The dye can be in solid, semi-solid or liquid form. The bath or solution is generally water that is heated to an elevated temperature either before, during or after the dye has been added. This temperature can range from between about 100° F. to over 300° F. Desirably, the temperature of the bath or solution is at least about 150 degrees. More desirably, the temperature of the bath or solution is at least about 180 degrees. Even more desirably, the temperature of the bath or solution is at least about 200 degrees. Most desirably, the temperature of the bath or solution is over 200 degrees. The fabric or material will remain in the bath or solution for a set period of time. The bath or solution can be agitated, if desired. The time period will vary depending on a number of factors, including but not limited to: the size of the vessel, the type of liquid making up the bath or solution, the amount and composition of the fabric or material being dyed, the temperature of the bath or solution, the color to which the fabric or material is to be dyed to, etc.

The stretchable loop material 58 has at least about 150% elongation in a first direction and has at least 50% elongation in a second direction. The first direction is aligned perpendicular or at 90 degrees to the second direction. The first direction can be the machine direction in which the material was produced and the second direction can be the cross-direction of the material. The machine direction is aligned perpendicular to the cross-direction. Desirably, the stretchable loop material 58 has an elongation of at least about 175% in the first direction. More desirably, the stretchable loop material 58 has an elongation of at least about 200% in the first direction. Even more desirably, the stretchable loop material 58 has an elongation of at least about 220% in the first direction. Most desirably, the stretchable loop material 58 has an elongation of over 220% in the first direction.

The stretchable loop material 58 has at least about 50% elongation in the second direction. Desirably, the stretchable loop material 58 has an elongation of about 55% in the second direction. More desirably, the stretchable loop material 58 has at least about 60% elongation in the second direction. Even more desirably, the stretchable loop material 58 has an elongation of about 65% in the second direction. Most desirably, the stretchable loop material 58 has an elongation of over 70% in the second direction.

Still referring to FIGS. 9 and 11, the stretchable loop material 58 is secured to the first end 50, the second end 52, and to a portion of the first and second sides, 54 and 56 respectively, of the absorbent structure 42. The securement can be by any method known to those skilled in the art. For example, the securement can be accomplished using a mechanical connection like sewing with thread, by a chemical bond, such as using an adhesive, a co-adhesive, or glue, or by using heat, pressure, or a combination of heat and pressure. Such securement forms the disposable undergarment 10″.

The disposable undergarment 10″ has a front portion 60, a back portion 62, and a crotch portion 64 which joins the front portion 60 to the back portion 62. The disposable undergarment 10″ also has a pair of lateral sides 66 and 68 securing a portion of the front portion 60 to a portion of the back portion 62. The disposable undergarment 10″ further has a hip opening 70. By “hip” it is meant the laterally projecting prominence of the pelvis or pelvic region from the waist to the thigh. The hip opening 70 surrounds the pelvic region when secured to a human torso. The machine direction of the stretchable loop material 58 should lie parallel to the hip opening 70 when the stretchable loop material 58 is formed into the disposable undergarment 10″. This means that the stretchable loop material 58 will have its maximum stretch or elongation in the circumferential direction around the hip of the person wearing the disposable undergarment 10″.

It should be noted that the hip opening 70 of the disposable undergarment 10″ is situated at a lower position on the human torso than would a disposable undergarment having a waist opening. When the disposable undergarment 10″ is positioned on a person's lower torso, the hip opening 70 will be located below the person's bellybutton. By “bellybutton” it is meant the navel; the umbilicus.

The disposable undergarment 10″ also has a pair of spaced apart leg openings 72 and 74. The disposable undergarment 10″ further has an interior surface 76.

Referring to FIG. 9, one can clearly see that the height h of the first and second members, 78 and 80 respectively, at their point of attachment to one another is rather limited. The height h is measured between the hip opening 70 and one of the pair of leg openings 72 and 74. This height h is small because the disposable undergarment 10″ is designed to fit around the pelvic region of a person and does not extend upward to the waist of the person. It is sometimes referred to as a “hipster” or “low rider”. Typically, this height h dimension is about 4.5 inches or less. Desirably, this height h dimension is less than about 4 inches. More desirably, this height h dimension is less than about 3.75 inches. Even more desirably, this height h dimension is less than about 3.5 inches. Most desirably, this height h dimension ranges from between about 2 inches to about 4 inches.

It should be noted that the stretchable loop material 58 forms at least about 50% of the exterior surface 40 of the disposable undergarment 10″. Desirably, the stretchable loop material 58 forms at least about 60% of the exterior surface 40 of the disposable undergarment 10″. More desirably, the stretchable loop material 58 forms at least about 70% of the exterior surface 40 of the disposable undergarment 10″. Even more desirably, the stretchable loop material 58 forms at least about 80% of the exterior surface 40 of the disposable undergarment 10″. Most desirably, the stretchable loop material 58 forms from between about 50% to about 100% of the exterior surface 40 of the disposable undergarment 10″.

Still referring to FIGS. 9 and 11, each of the pair of lateral sides 66 and 68 is bifurcated from the hip opening 70 to one of the pair of leg openings 72 and 74 to form a first member 78 and a second member 80. By “bifurcated” it is meant to divide or separate into two parts. In FIG. 9, one can clearly see that the bifurcation is a vertical slit or cut. Other ways of separating the first member 78 from the second member 80 in each of the pair of lateral sides 66 and 68 are known to those skilled in the art. Each of the first and second members, 78 and 80 respectively, has an inner surface 82 and an outer surface 84. A pair of attachment members 86, 86 is secured to the outer surface 84 of each of the first members 78, 78. Each of the pair of attachment members 86, 86 is formed from a hook material. The hook material can be similar or identical to VELCRO® which is a registered trademark of Velcro Industries B. V. LLC, having an office at Castorweg 22-24 Curacao, Netherlands. Each of the pair of attachment members 86, 86 is capable of engaging with the inner surface 82 of one of the second members 80, 80 to enable the disposable undergarment 10″ to acquire a closed configuration, see FIG. 11. Separation of the pair of attachment members 86, 86 from each of the second members 80, 80 enables the disposable undergarment 10″ to acquire an open configuration, as shown in FIG. 9.

Referring now to FIG. 12, a disposable undergarment 11 is shown which is similar in construction to the disposable undergarment 10″ except that at least two elastic strands 88, 88 are positioned above the first and second ends, 50 and 52 respectively, of the absorbent structure 42. The at least two elastic strands 88, 88 are located in the stretchable loop material 58. The at least two elastic strand 88, 88 are aligned parallel to the hip opening 70. The at least two elastic strands 88, 88 function to assist the stretchable loop material 58 in holding the disposable undergarment 11 adjacent to the hips of the person wearing the disposable undergarment 11. The at least two elastic strands 88, 88 can include 3, 4, 5, 6, 7, 8, 9, 10 or more elastic strands 88. The at least two elastic strands 88, 88 can be positioned at various distances apart. If three or more elastic strands 88 are utilized, they can be spaced an equal distance apart or they can be arranged in a random fashion.

It should be noted that in this embodiment, no additional elastic strands 88, 88 are present in the pair of lateral sides 66 and 68.

Referring now to FIG. 13, a third embodiment of a disposable undergarment 11′ is shown which varies from the embodiment depicted in FIG. 12 in that at least two elastic strands 88, 88 extend completely around the hip opening 70. The at least two elastic strands 88, 88 are located in the stretchable loop material 58. The at least two elastic strands 88, 88 are aligned parallel to the hip opening 70. The at least two elastic strands 88, 88 function to assist the stretchable loop material 58 in holding the disposable undergarment 11′ adjacent to the hips of the person wearing the disposable undergarment 11′. The at least two elastic strands 88, 88 can include 3, 4, 5, 6, 7, 8, 9, 10 or more elastic strands 88. The at least two elastic strands 88, 88 can be positioned at various distances apart. If three or more elastic strands 88 are utilized, they can be spaced an equal distance apart or they can be arranged in a random fashion.

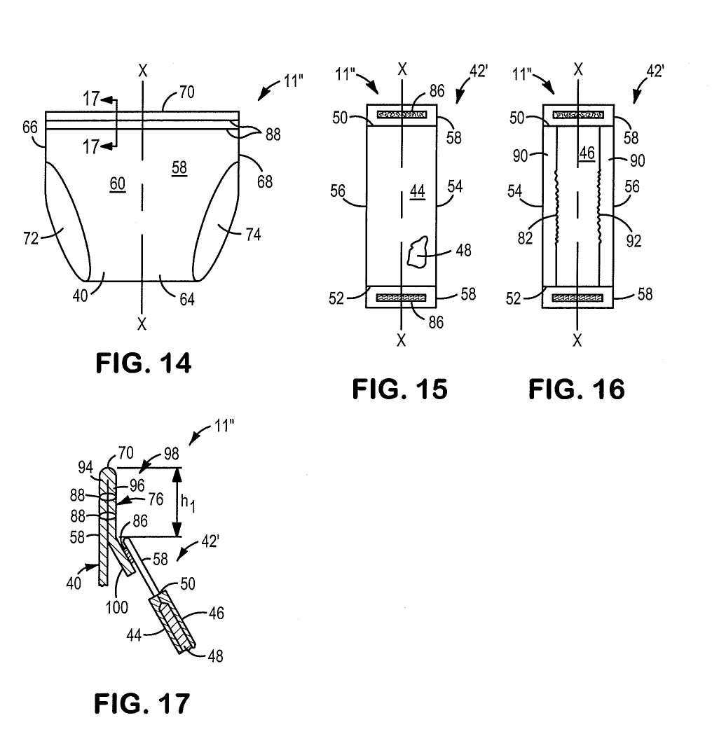

Referring now to FIGS. 14-16 a fourth embodiment of a disposable undergarment 11″ is shown. In this embodiment, is shown in FIG. 14, the entire exterior surface 40 of the disposable undergarment 11′ is formed from a stretchable loop material 58. The disposable undergarment 11′ has a hip opening 70 and at least two elastic strands 88, 88 located in the stretchable loop material 58. The at least two elastic strands 88, 88 are aligned parallel to the hip opening 70. The at least two elastic strands 88, 88 function to assist the stretchable loop material 58 in holding the disposable undergarment 11″ adjacent to the hips of the person wearing the disposable undergarment 11″. The at least two elastic strands 88, 88 can include 3, 4, 5, 6, 7, 8, 9, 10 or more elastic strands 88. The at least two elastic strands 88, 88 can be positioned at various distances apart. If three or more elastic strands 88 are utilized, they can be spaced an equal distance apart or they can be arranged in a random fashion.

Referring to FIGS. 15 and 16, the disposable undergarment 11″ also includes a removable absorbent structure 42′. The back of the absorbent structure 42′ is depicted in FIG. 15 and the front of the absorbent structure 42′ is depicted in FIG. 16. The absorbent structure 42′ includes a liquid impermeable outer cover 44, see FIG. 15, a liquid-permeable bodyside liner 46 secured to the outer cover 44, see FIG. 16, and an absorbent 48 sandwiched therebetween. The absorbent structure 42′ also has a first end 50, a second end 52, a first side 54 and a second side 56. A stretchable loop material 58 is secured to the first and second ends, 50 and 52 respectively, of the absorbent structure 42′. A pair of attachment members 86, 86 is secured to the stretchable loop material 58 on the back side of the absorbent structure 42′. Each of the pair of attachment members 86, 86 is formed from a hook material. The hook material can be similar or identical to VELCRO® which is a registered trademark of Velcro USA Inc., having an office at 406 Brown Avenue, Manchester, N.H. 03103. The pair of attachment members 86, 86 permit the absorbent structure 42′ to be removably attached to an inner surface of the stretchable loop material 58. The stretchable loop material 58 acts as the loop component of the hook and loop fastener.

The disposable undergarment 11″ is sometimes referred to as a “semi-disposable undergarment” since the absorbent structure 42′ can be discarded when soiled but the remainder of the undergarment 11″, the portion constructed of the stretchable loop material 58 can be reused multiple times. The stretchable loop material 58 can also be laundered up to twenty times before it has to be replaced.

Referring to FIG. 16, one can see that a pair of barrier cuffs 90, 90 is present on the front of the absorbent structure 42′. Each of the barrier cuffs 90, 90 contains one or more elastic strands 92 which cause the pair of barrier cuffs 90, 90 to acquire a vertical arrangement above the liquid-permeable bodyside liner 46. The pair of barrier cuffs 90, 90 assist in trapping and retaining bodily waste, such as waste matter discharged from the large intestine, in the absorbent structure 42′.

It should be understood that the pair of barrier cuffs 90, 90 are optional, and do not have to be present, if desired.

Referring now to FIG. 17, one way of securing the absorbent structure 42′ to the interior surface 76 of the disposable undergarment 11″ is depicted. As shown, the stretchable loop material 58 is folded upon itself at the hip opening 70 to form two distinct layers 94 and 96. The layer 94 is located adjacent to the exterior surface 40 and the layer 96 is located adjacent to the interior surface 76. The at least two elastic strands 88, 88 are secured across the two layers 94 and 96 to form a hip band 98. The hip band 98 has a height h₁ which can vary in height. A height h₁ of the hip band 98 can range from between about 0.5 inches to about 2 inches. Desirably, the height h₁ of the hip band 98 is at least about 0.75 inches. More desirably, the height h₁ of the hip band 98 is at least about 1 inch. Even more desirably, the height h₁ of the hip band 98 is at least about 1.25 inches. Most desirably, the height h₁ of the hip band 98 is at least about 1.5 inches.

A lower portion of the layer 96 is unattached to the layer 94. This lower portion of the layer 96 angles downward and/or away from the layer 94, below the hip band 98, and creates a flap 100. The flap 100 is formed from the stretchable loop material 58 and therefore serves as the loop portion of a hook and loop attachment. The flap 100 is located adjacent to the interior surface 76 of the disposable undergarment 11″, approximate the front thereof. A similar flap can be located adjacent to the interior surface 76 of the disposable undergarment 11″, approximate the rear thereof. The attachment member 86, located approximate the first end 50 of the absorbent structure 42′, see FIG. 15, can be secured to the flap 100. This action will secure the first end 50 of the absorbent structure 42′ to the disposable undergarment 11″. The attachment member 86, located approximate the second end 52 of the absorbent structure 42′, see FIG. 15, can be secured to a second flap (not shown). This action will secure the second end 52 of the absorbent structure 42′ to the disposable undergarment 11″. With both the first and second ends, 50 and 52 respectively, of the absorbent structure 42′ secured to the disposable undergarment 11″, the absorbent structure 42′ will be securely held in placed on the interior surface 76 of the disposable undergarment 11″.

It should be understood that various other arrangements can be utilized to securing the absorbent structure 42′ to the interior surface 76 of the disposable undergarment 11″.

### Method of Securing the Disposable Undergarment to a Person's Torso

A method of securing the disposable undergarment 10″ about a person's lower torso will now be described. The disposable undergarment 10″ has an absorbent structure 42 which includes a liquid-impermeable outer cover 44, a liquid-permeable bodyside liner 46 secured to the outer cover 44, and an absorbent 48 positioned between the outer cover 44 and the bodyside liner 46. The absorbent structure 42 has a first end 50, a second end 52, a first side 54 and a second side 56. The disposable undergarment 10″ also includes a stretchable loop material 58 having at least about 150% elongation in a first direction and at least about 50% elongation in a second direction. The first direction is aligned perpendicular or at 90 degrees to the second direction. The stretchable loop material 58 is secure to the first end 50, the second end 52, and to a portion of the first and second sides, 54 and 56 respectively, of the absorbent structure 42. The disposable undergarment 10″ has a front portion 60, a back portion 62, and a crotch portion 64 which joins the front portion 60 to the back portion 62.

The disposable undergarment 10″ also includes a pair of lateral sides 66 and 68 securing a portion of the front portion 60 to a portion of the back portion 62. The disposable undergarment 10″ further includes a hip opening 70, a pair of spaced apart leg openings 72 and 74 and an interior surface 76 and an exterior surface 40. The stretchable loop material 58 forms at least about 50% of the exterior surface 40 of the disposable undergarment 10″. Each of the pair of lateral sides 66 and 68 is bifurcated from the hip opening 70 to one of the pair of leg openings 72 and 74 to form a first member 78 and a second member 80. Each of the first and second members, 78 and 80 respectively, has an inner surface 82 and an outer surface 84. A pair of attachment members 86, 86, each formed from a hook material, is secured to the outer surface 84 of one of the first members 78, 78. Each of the pair of attachment members 86, 86 is capable of engaging with the inner surface 82 of one of the second members 80, 80 to enable the disposable undergarment 10″ to acquire a closed configuration. Separation of the pair of attachment members 86, 86 from each of the second members 80, 80 enables the disposable undergarment 10″ to acquire an open configuration.

The method includes the steps of releasing the pair of attachment members 86, 86 from the second members 80, 80 and allowing the disposable undergarment 10″ to acquire the open configuration with the interior surface 76 facing upward. The inner surface 82 of the back portion 62 is then positioned adjacent to a person's buttock. The front portion 60 of the disposable undergarment 10″ is then grasped and moved so that it is positioned adjacent to a person's stomach, below their bellybutton, with the crotch portion 64 covering the person's crotch area. Lastly, the first and second members, 78 and 80 respectively, of each of the pair of lateral sides 66 and 68 are sequentially stretched such that they overlap and engage each of the attachment members 86, 86 to the respective second member 80, 80 so that the disposable undergarment 10″ acquires a closed configuration and is held secure to the person's lower torso.

The method further includes sequentially releasing each of the second members 80, 80 from each of the first members 78, 78 in each of the pair of lateral sides 66 and 68 and allowing the disposable undergarment 10″ to acquire an open configuration which permits the disposable undergarment 10″ to be removed from the person's lower torso.

It should be understood that the disposable undergarment 10″ can be secured to a person's lower torso when the person is standing or is lying on their back. Most bedridden patients, who are lying down, need a caregiver to position the disposable undergarment 10″ on their lower torso. The caregiver would also be needed to remove the disposable undergarment 10″ once it becomes soiled with bodily waste and to replace the disposable undergarment 10″ with a new disposable undergarment 10″.

### Method of Disposing of a Soiled Disposable Undergarment

A method of disposing of a soiled disposable undergarment 10″ will now be described. The disposable undergarment 10″ has an absorbent structure 42 which includes a liquid-impermeable outer cover 44, a liquid-permeable bodyside liner 46 secured to the outer cover 44, and an absorbent 48 positioned between the outer cover 44 and the bodyside liner 46. The absorbent structure 42 has a first end 50, a second end 52, a first side 54 and a second side 56. The disposable undergarment 10″ also includes a stretchable loop material 58 having at least about 150% elongation in a first direction and at least about 50% elongation in a second direction. The first direction is aligned perpendicular or at 90 degrees to the second direction. The stretchable loop material 58 is secure to the first end 50, the second end 52, and to a portion of the first and second sides, 54 and 56 respectively, of the absorbent structure 42. The disposable undergarment 10″ has a front portion 60, a back portion 62, and a crotch portion 64 which joins the front portion 60 to the back portion 62.

The disposable undergarment 10″ also includes a pair of lateral sides 66 and 68 securing a portion of the front portion 60 to a portion of the back portion 62. The disposable undergarment 10″ further includes a hip opening 70, a pair of spaced apart leg openings 72 and 74, an interior surface 76 and an exterior surface 40. The stretchable loop material 58 forms at least about 50% of the exterior surface 40 of the disposable undergarment 10″. Each of the pair of lateral sides 66 and 68 is bifurcated from the hip opening 70 to one of the pair of leg openings 72 and 74 to form a first member 78 and a second member 80. Each of the first and second members, 78 and 80 respectively, has an inner surface 82 and an outer surface 84. A pair of attachment members 86, 86, each formed from a hook material, is secured to the outer surface 84 of one of the first members 78, 78. Each of the pair of attachment members 86, 86 is capable of engaging with the inner surface 82 of one of the second members 80, 80 to enable the disposable undergarment 10″ to acquire a closed configuration. Separation of the pair of attachment members 86, 86 from each of the second members 80, 80 enables the disposable undergarment 10″ to acquire an open configuration.

The method includes the steps of sequentially releasing each of the second members 80, 80 from each of the first members 78, 78 in each of the pair of lateral sides 66 and 68 after the disposable undergarment 10″ has becomes soiled with human waste and/or body fluid. The disposable undergarment 10″ is then allowed to acquire an open configuration such that the soiled disposable undergarment 10″ can be removed from the person's lower torso. The method further includes rolling or folding the front portion 60 of the soiled disposable undergarment 10″ inward and back toward the back portion 62. Each of the second members 80, 80 is then overlapped to encase the rolled or folded soiled disposable undergarment 10″. Lastly, the second members 80, 80 are tied together to retain the soiled disposable undergarment 10″ in a sanitary fashion.

While the particular preferred embodiments have been shown and described, it is obvious to those skilled in the art that changes and modifications may be made without departing from the teaching of the disclosure. The matter set forth in the foregoing description and accompanying drawings is offered by way of illustration only and not as limitation. The actual scope of the disclosure is intended to be defined in the following claims when viewed in their proper perspective based on the related art.

We claim:

1. A disposable undergarment having an exterior surface, and said undergarment is capable of being positioned on and being removed from a human torso in either a dosed configuration or in an open configuration, said disposable undergarment comprising:
(a) an absorbent structure including a liquid-impermeable outer cover, a liquid-permeable bodyside liner secured to said outer cover, and an absorbent positioned between said outer cover and said bodyside liner, and said absorbent structure having a first end, a second end, a first side and a second side;
(b) a stretchable loop material dyed to a color darker than white and having at least about 150% elongation in a first direction and at least about 50% elongation in a second direction, said stretchable loop material being secure to said first end, said second end and to a portion of said first and second sides of said absorbent structure to form said disposable undergarment having a front portion, a back portion and a crotch portion which joins said front portion to said back portion, a pair of lateral sides securing a portion of said front portion to a portion of said back portion, a hip opening, a pair of spaced apart leg openings, an interior surface and an exterior surface, and said stretchable loop material forms at least about 50% of said exterior surface of said disposable undergarment;
(c) each of said pair of lateral sides being bifurcated from said hip opening to one of said pair of leg openings to form a first member and a second member, and each of said first and second members having an inner surface and an outer surface; and
(d) a pair of attachment members each formed from a hook material, each of said pair of attachment members being secured to said outer surface of one of said first members, each of said pair of attachment members capable of engaging with said inner surface of one of said second members to enable said disposable undergarment to acquire said closed configuration, and separation of said pair of attachment members from each of said second members enabling said disposable undergarment to acquire said open configuration.

2. The disposable undergarment of claim 1 wherein the vertical distance between said hip opening and each of said pair of leg openings is less than about 4 inches, and said stretchable loop material has a basis weight of from between about 15 gsm to about 175 gsm, and a density of from between about 0.02 g/cm3 to about 0.12 g/cm3.

3. The disposable undergarment of claim 1 wherein said stretchable loop material, positioned above said first and second ends of said absorbent structure, contains at least two elastic strands which are aligned parallel to said hip opening.

4. The disposable undergarment of claim 1 wherein said stretchable loop material is a stitch-bonded nonwoven fabric and at least two elastic strands extend completely around said hip opening and said two elastic strands are aligned parallel to said hip opening.

5. The disposable undergarment of claim 4 wherein said stitch-bonded nonwoven fabric can be washed and dried at least twenty times before losing its stretchability.

6. The disposable undergarment of claim 1 wherein said stretchable loop material forms at least about 60% of said exterior surface area of said disposable undergarment, and said stretchable loop material is dyed to a color darker than white.

7. The disposable undergarment of claim 6 wherein said stretchable loop material forms at least about 70% of said exterior surface area of said disposable undergarment.

8. The disposable undergarment of claim 6 wherein said stretchable loop material forms at least about 80% of said exterior surface area of said disposable undergarment.

9. The disposable undergarment of claim 6 wherein said stretchable loop material forms from between about 50% to 100% of said exterior surface area of said disposable undergarment.

10. A disposable undergarment having an exterior surface area, and said undergarment is capable of being positioned on and being removed from a human torso in either a dosed configuration or in an open configuration, said disposable undergarment comprising:
(a) an absorbent structure including a liquid-impermeable outer cover, a liquid-permeable bodyside liner secured to said outer cover, and an absorbent positioned between said outer cover and said bodyside liner, and said absorbent structure having a first end, a second end, a first side and a second side;
(b) a stretchable loop material dyed to a color darker than white and having at least about 175% elongation in a first direction and at least 60% elongation in a second direction, said stretchable loop material being secure to said first end, said second end and to a portion of said first and second sides of said absorbent structure to form said disposable undergarment having a front portion, a back portion and a crotch portion which joins said front portion to said back portion, a pair of lateral sides securing a portion of said front portion to a portion of said back portion, a hip opening, a pair of spaced apart leg openings, an interior surface and an exterior surface, and said stretchable loop material forms at least about 60% of said exterior surface area of said disposable undergarment;
(c) each of said pair of lateral sides being bifurcated from said hip opening to one of said pair of leg openings to form a first member and a second member, and each of said first and second members having an inner surface and an outer surface; and
(d) a pair of attachment members each formed from a hook material, each of said pair of attachment members being secured to said outer surface of one of said first members, each of said pair of attachment members capable of engaging with said inner surface of one of said second members to enable said disposable undergarment to acquire said closed configuration, and separation of said pair of attachment members from each of said second members enabling said disposable undergarment to acquire said open configuration.

11. The disposable undergarment of claim 10 wherein said first direction of elongation in said stretchable loop material is in a machine direction and said second direction of elongation is in a perpendicular cross direction, and said machine direction of said stretchable loop material lies parallel to said hip opening when formed into said disposable undergarment.

12. The disposable undergarment of claim 10 wherein said stretchable loop material forms from between about 50% to 100% of said exterior surface area of said disposable undergarment.

13. The disposable undergarment of claim 10 wherein said stretchable loop material has a basis weight of greater than 100 gsm, and a density greater than about 0.05 g/cm3.

14. The disposable undergarment of claim 10 wherein said stretchable loop material has at least about 200% elongation in said first direction.

15. The disposable undergarment of claim 10 wherein said stretchable loop material has over 220% elongation in said first direction.

16. A method of securing a disposable undergarment about a person's lower torso, said disposable undergarment having a liquid-impermeable outer cover, a liquid-permeable bodyside liner secured to said outer cover, an absorbent positioned between said outer cover and said bodyside liner to form an absorbent structure having a first end, a second end, a first side and a second side, a stretchable loop material having at least about 150% elongation in a first direction and at least about 50% elongation in a second direction, said stretchable loop material being secure to said first end, said second end and to a portion of said first and second sides of said absorbent structure to form said disposable undergarment having a front portion, a back portion and a crotch portion which joins said front portion to said back portion, a pair of lateral sides securing a portion of said front portion to a portion of said back portion, a hip opening, a pair of spaced apart leg openings, an interior surface and an exterior surface, and said stretchable loop material forms at least about 50% of said exterior surface of said disposable undergarment, each of said pair of lateral sides being bifurcated from said hip opening to one of said pair of leg openings to form a first member and a second member, and each of said first and second members having an inner surface and an outer surface, and a pair of attachment members each formed from a hook material, each of said pair of attachment members being secured to said outer surface of one of said first members, each of said pair of attachment members capable of engaging with said inner surface of one of said second members to enable said disposable undergarment to acquire said closed configuration, and separation of said pair of attachment members from each of said second members enabling said disposable undergarment to acquire said open configuration, said method comprising the steps of:
(a) releasing said pair of attachment members and allowing said disposable undergarment to acquire said open configuration with said interior surface facing upward;
(b) positioning said interior surface of said back portion adjacent to a person's buttock;
(c) grasping and moving said front portion of said disposable undergarment adjacent to a person's stomach, below their bellybutton, with said crotch portion covering the person's crotch area; and
(d) sequentially stretching and overlapping said first and second members of each of said pair of lateral sides and engaging each of said attachment members to said respective second member so that said disposable undergarment acquires a closed configuration and is held secure to the person's lower torso.

17. The method of claim 16 further comprising the step of sequentially releasing each of said second members from each of said first members in each of said pair of lateral sides and allowing said disposable undergarment to acquire an open configuration which permits said disposable undergarment to be removed from the person's lower torso.

18. The method of claim 16 wherein said disposable undergarment is secured to a person's lower torso when said person is standing.

19. The method of claim 16 wherein said disposable undergarment is secured to a person's lower torso when said person is lying on their back.

20. A method of disposing of a soiled disposable undergarment, said disposable undergarment having a liquid-impermeable outer cover, a liquid-permeable bodyside liner secured to said outer cover, an absorbent positioned between said outer cover and said bodyside liner to form an absorbent structure having a first end, a second end, a first side and a second side, a stretchable loop material having at least about 150% elongation in a first direction and at least about 50% elongation in a second direction, said stretchable loop material being secure to said first end, said second end and to a portion of said first and second sides of said absorbent structure to form said disposable undergarment having a front portion, a back portion and a crotch portion which joins said front portion to said back portion, a pair of lateral sides securing a portion of said front portion to a portion of said back portion, a hip opening, a pair of spaced apart leg openings, an interior surface and an exterior surface, and said stretchable loop material forms at least about 50% of said exterior surface of said disposable undergarment, each of said pair of lateral sides being bifurcated from said hip opening to one of said pair of leg openings to form a first member and a second member, and each of said first and second members having an inner surface and an outer surface, and a pair of attachment members each formed from a hook material, each of said pair of attachment members being secured to said outer surface of one of said first members, each of said pair of attachment members capable of engaging with said inner surface of one of said second members to enable said disposable undergarment to acquire said closed configuration, and separation of said pair of attachment members from each of said second members enabling said disposable undergarment to acquire said open configuration, said method comprising the steps of:
(a) sequentially releasing each of said second members from each of said first members in each of said pair of lateral sides after said disposable undergarment has becomes soiled with human waste or body fluid;
(b) allowing said disposable undergarment to acquire an open configuration and removing said soiled disposable undergarment from the person's lower torso;
(c) rolling or folding said front portion of said soiled disposable undergarment inward and back toward said back portion;
(d) overlapping each of said second members to encase said rolled or folded soiled disposable undergarment; and
(e) tying said second members together to retain said soiled disposable undergarment in a sanitary fashion.
